(19) United States
(12) Patent Application Publication
(10) Pub. No.: US 2025/0209202 A1
(43) Pub. Date: Jun. 26, 2025

(54) COMPUTER SYSTEM AND METHODS FOR AUGMENTING A GRAPHICAL USER INTERFACE

(71) Applicant: Io Health Technologies, LLC, Pasadena, CA (US)

(72) Inventors: David A. Bell, La Canada, CA (US); Hassaan Raza, Ilford (GB)

(21) Appl. No.: 18/966,849

(22) Filed: Dec. 3, 2024

Related U.S. Application Data
(60) Provisional application No. 63/607,965, filed on Dec. 8, 2023.

(51) Int. Cl. G06F 21/62; G16H 10/60

(52) U.S. Cl. CPC G06F 21/6245; G16H 10/60

(57) ABSTRACT

According to embodiments, a computer method for augmenting data includes keylogging a user interacting with a fillable form and/or capturing an at least momentary image of the fillable form, determining a format or indicia corresponding to a field of the fillable form, and causing display of the format or indicia over or in place of and registered to the field of the fillable form. Embodiments may enable protection of personally identifiable information and/or protected health information (PHI) from unauthorized eyes. Embodiments may identify errors in data entry. Embodiments may guide a user through a preferred form filling process.

### CROSS REFERENCE TO RELATED APPLICATION

The present application claims priority benefit from U.S. Provisional Patent Application No. 63/607,965 entitled “COMPUTER SYSTEM AND METHOD FOR AUGMENTING A GRAPHICAL USER INTERFACE,” filed Dec. 8, 2023 (docket number 3094-001-02), which to the extent not inconsistent with the disclosure herein, is incorporated by reference.

### BACKGROUND

The Health Insurance Portability and Accountability Act (HIPAA) of 1996 has, in one aspect, generally been distilled down to HIPAA-restricted health management computer (HHM) systems for handling protected health information (PHI). While advances have been made with respect to device portability, entrenched system providers have tended to maintain “legacy” user interfaces with respect to uniqueness, and non-compatibility with one another. This aspect tends to add training requirements and/or require particularly high attention to operate effectively and efficiently.

### SUMMARY

In an aspect, the inventors discovered that HIPAA-restricted health management computer (HHM) interfaces may be susceptible to exposing protected health information (PHI) on unguarded electronic displays. Example application types include electronic medical record systems, such as EPIC, Cerner, Allscripts, Home Care Home Base, WellSky and many others; HIPAA-compliant messaging systems such as TigerConnect; medical billing systems such as Zirmed or Kareo; and chart review systems such as SHP OASIS Scrubbing.

According to embodiments, systems and computer methods described herein provide masking of sensitive information without regard to particular API characteristics or access to communication tokens.

In an aspect, the inventors discovered that HHM interfaces may lack commonality with respect to how patient forms are filled out. These diverse user interfaces may pose training or familiarity challenges. According to embodiments, systems and computer methods described herein may provide user guidance corresponding to optimized data entry and/or data access workflow.

As used herein, the term “screen scrape” and the like refers to a colloquialism that the inventors intend to span plural optional graphical display access technologies (described in more detail below). According to an embodiment, a computer method for graphically augmenting a graphical user interface of a HIPAA-restricted computer application includes the steps of performing screen scraping of an image layer produced by a computer application to create a screen-scraped image; comparing the screen-scraped image to a corresponding reference database to determine a conditional state corresponding to a portion of the image layer; and displaying, via an at least partially transparent layer over the image layer produced by the computer application, one or more indicia responsive to the conditional state, the indicia being registered to the portion of the image layer.

According to an embodiment, a computer system for providing user-guidance includes an electronic display, driven by a computer, configured to display a user interface including at least a portion of a fillable form produced by a HIPAA-restricted medical application; a video capture interface configured to capture video data including at least a portion of the displayed fillable form; and a microprocessor configured to execute computer-readable instructions. The computer-readable instructions correspond to an image optimizer configured to receive the captured video data from the video capture interface, the image optimizer being configured to determine context and content of the at least the portion of the fillable form and thin the video data to include (such as only include) at least one graphical data field of the fillable form; a data reader configured to receive the at least one graphical data field from the image optimizer and perform optical character recognition to convert the at least one graphical data field to at least one corresponding character-type (e.g., ASCII or Unicode) data field; and a data analyzer configured to receive the at least one data field from the data reader, access a database carrying corresponding data field information, compare the at least one data field to the corresponding data field information, and output overlay data responsive to the at least one comparison. The computer system further includes a graphics processor configured to receive the overlay data from the data analyzer and to operate an embedded display engine configured to receive the overlay data and to drive the electronic display to display an overlay image adjacent to or over the at least one graphical data field on the electronic display.

According to an embodiment, a computer method includes the steps of capturing an image including a fillable form including at least one field at least nominally displayed on an electronic display; thinning the at least partially displayed image to reduce an amount of computer memory needed to hold information corresponding to the fillable form; matching the fillable form or thinned fillable form to a reference fillable form or reference thinned fillable form stored on a non-transitory computer readable medium; reading a format corresponding to at least one reference field in the reference fillable form or reference thinned fillable form corresponding to the at least one field; and causing display of the format registered to the at least one field superimposed over or adjacent to the at least one field.

According to embodiments, an edge-resident user interface application includes some or all of a video capture interface, an image optimizer, a data reader, a database, and a display engine configured to drive a selectively transparent or indicative image adjacent to or over a legacy data interface. The video capture interface, such as a video buffer reader, mirrored “display” receiver, or a camera, by way of example, receives an image displayed and/or intended for display on a user electronic display by an electronic medical records system (EMR). The image optimizer may obtain a target location in the display space, use a template or image analyzer to select masked pixels, and insert the mask into the image to be and/or displayed on the electronic display. Various layers are contemplated for introduction of the mask to the display list.

According to an embodiment, a computer method for augmenting a graphical user interface of a HIPAA-restricted computer application includes obtaining image data from a computer application corresponding to a first user interface, comparing data corresponding to the obtained image data to a corresponding reference database to determine a conditional state corresponding to at least a portion of the obtained image data, and electronically displaying one or more indicia responsive to the conditional state on an electronic display to the user.

According to an embodiment, a computer system for providing user-guidance includes an electronic display, driven by a computer, configured to display a user interface including at least a portion of a fillable form, such as a fillable form produced by a HIPAA-restricted medical application. A video capture interface may be configured to capture video data including at least a portion of the displayed fillable form. A microprocessor may be configured to execute computer-readable instructions corresponding to an image optimizer configured to receive the captured video data from the video capture interface, the image optimizer being configured to determine context and content of the at least the portion of the fillable form and thin the video data to include at least one graphical data field of the fillable form; a data reader configured to receive the at least one graphical data field from the image optimizer and perform optical character recognition to convert the at least one graphical data field to at least one corresponding character-type data field; and a data analyzer configured to receive the at least one character-type data field from the data reader, access a database carrying corresponding data field information, compare the at least one character-type data field to the corresponding data field information, and output augmentation data responsive to the at least one comparison; and a graphics processor configured to receive the augmentation data from the data analyzer and to operate an embedded display engine configured to receive the augmentation data and to drive the electronic display to display an augmentation image adjacent to or over the at least one graphical data field on the electronic display.

According to an embodiment, a computer method includes capturing keystrokes from a user interacting with a computer application; inferring an image including a fillable form including at least one field at least nominally displayed on an electronic display; matching the fillable form to a reference fillable form stored on a non-transitory computer readable medium; reading a format corresponding to at least one reference field in the reference fillable form; and causing display of the format registered to the at least one field.

According to an embodiment, a computer method for augmenting a graphical user interface of a HIPAA-restricted computer application includes obtaining user interface data from a computer application corresponding to a first user interface; comparing the obtained user interface data (or data derived from the obtained user interface data) to a corresponding reference database to determine a conditional state corresponding to at least a portion of the obtained user interface data; and electronically displaying one or more indicia responsive to the conditional state on an electronic display to the user.

### BRIEF DESCRIPTION OF THE DRAWINGS

FIG. 1A is a flow chart illustrating a computer method for augmenting a graphical user interface of a computer application, according to an embodiment.

FIG. 1B is a flow chart showing the computer method of FIG. 1A with additional detail, according to embodiments.

FIG. 1C is a flow chart showing the computer method of FIG. 1A with additional detail, according to other embodiments.

FIG. 2A is a diagram illustrating an example of an application display screen, according to an embodiment.

FIG. 2B is a diagram illustrating an example of an augmented application display screen, according to an embodiment.

FIG. 3A is a diagram illustrating an example of an application display screen, according to an embodiment.

FIG. 3B is a diagram illustrating an example of an augmented application display screen, according to an embodiment.

FIG. 4A is a diagram illustrating an example of an augmented application display screen, according to an embodiment.

FIG. 4B is a diagram illustrating an example of an augmented application display screen, according to an embodiment.

FIG. 5 is a diagram of a computer system configured to augment a graphical user interface output by a computer program, according to an embodiment.

FIG. 6 is a flowchart showing a computer method for causing display of a format relative to at least one field in a fillable form, according to an embodiment.

### DETAILED DESCRIPTION

In the following detailed description, reference is made to the accompanying drawings, which form a part hereof. In the drawings, similar symbols typically identify similar components, unless context dictates otherwise. Other embodiments may be used and/or other changes may be made without departing from the spirit or scope of the disclosure.

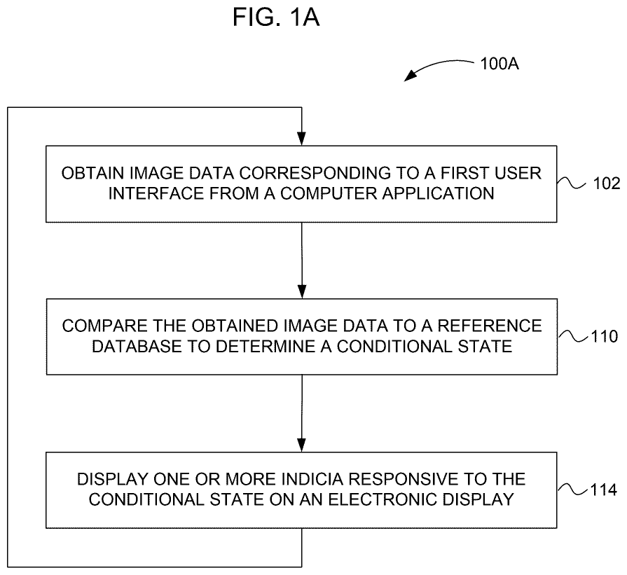

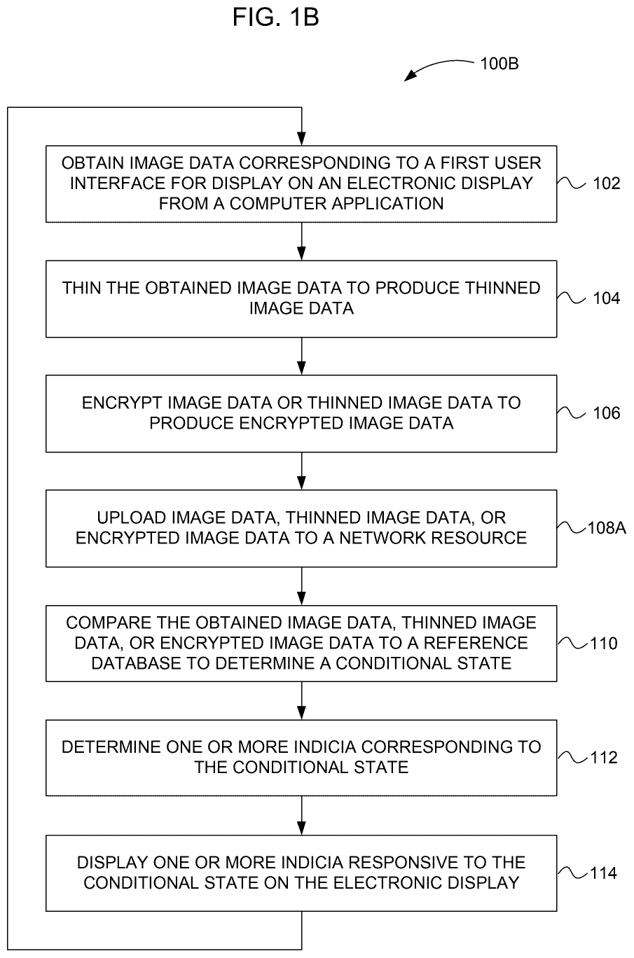

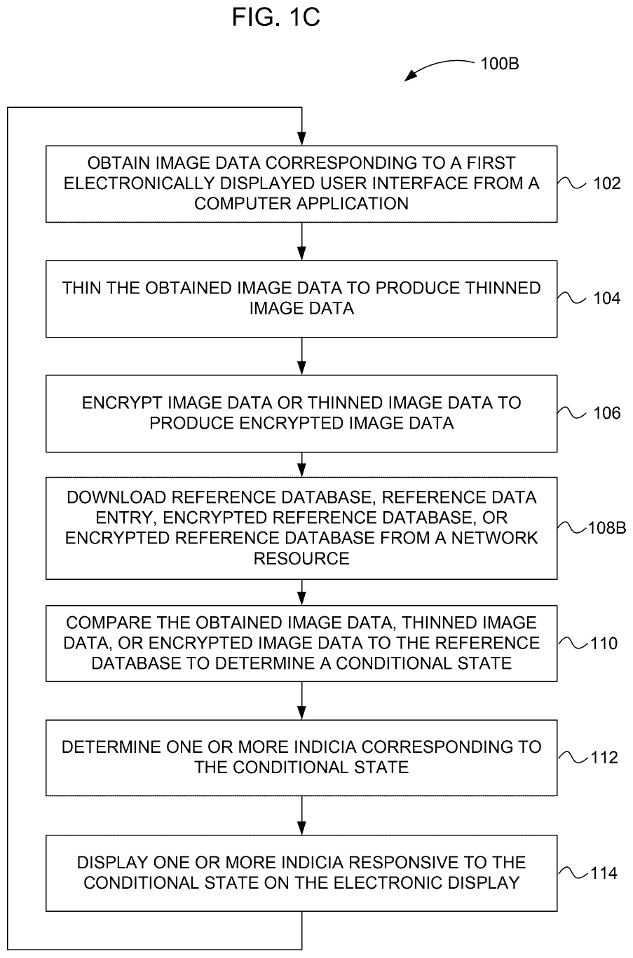

FIG. 1A is a flowchart illustrating a computer method 100A for augmenting a graphical user interface (GUI) of a computer application, such as a HIPAA-restricted computer application, according to an embodiment. As used herein, reference numbers 100A, 100B, and 100C are used to refer to various embodiments described in FIGS. 1A, 1B, and 1C, except where context dictates otherwise. Accordingly, references to the computer method 100 (or 100A, 100B, 100C) will be understood to any of FIG. 1A, 1B or 1C, except where context indicates otherwise.

The term HIPAA-restricted computer application is used herein to refer to a computer application containing medical or health information including protected health information (PHI) related to one or more patients used by a person working in the healthcare industry. Examples of such HIPAA-restricted applications may include electronic health record systems, email clients, efax applications, web browsers, billing applications, bill coding applications, messaging applications, spreadsheet applications, text editing applications, insurance forms, living wills, etc. The inventors contemplate applications focused on health industry-related applications represent a specific example of genera that extend across B2B and B2C computer methods, as well as non-transitory computer readable media carrying corresponding computer instructions.

As used herein the terms augmentation, augmentation data, and the like are to be understood as material added to or replacing material nominally presented by a computer application, such as a HIPAA-restricted computer application, according to computer methods and systems described herein.

Hardware on which computer applications suitable for augmentation operate, and accordingly on which embodiments of GUI augmentation operate, is contemplated to span a range of platforms including computers, laptop computers, client-server systems, tablet computers, mobile phones, etc.

Computer methods and systems described herein may apply to augmenting a computer application that is running on a virtual machine such as Citrix Desktop, as well as other environments.

As used herein, the term obtaining image data and the like is to be understood to refer both to bitmapped image data (such as pixel values) as well as to instructions used by an operating system or environment to construct a bitmap to be used as image data, except where context indicates otherwise. Similarly, the term comparing image data and the like is to be understood to refer to comparing actual image data, comparing instructions used by an operating system or environment to construct a bitmap to be used as image data, as well as to derivatives of the image data, except where context indicates otherwise.

According to an embodiment, in reference to FIG. 1A, a computer method 100 for augmenting a graphical user interface of a HIPAA-restricted computer application, includes, in step 102, obtaining image data from a computer application corresponding to a first user interface, such as for display on an electronic display by the computer application. The inventors contemplate computer methods and apparatuses disclosed more specifically herein may be used in conjunction with image data from other types of computer applications to obtain corresponding benefits and more, according to other types of computer application scope. Colloquially, obtaining image data in step 102 may be referred to as performing screen scraping of image display data. Such image display data may optionally be transitory such that a computer application GUI screen is captured then replaced by augmentation image data substantially without a user receiving a usable view of the non-augmented computer application GUI screen. In other embodiments, application image display data may be obtained without physical output of the non-augmented application GUI screen.

Various approaches and hardware embodiments for obtaining computer application display data are described in greater detail below.

Proceeding generally to step 110, the obtained image data is compared to a corresponding reference database to determine a conditional state corresponding to at least a portion of the obtained image data. For example, step 110 may include generating an output state responsive to Boolean (and optionally, Bayesian) processing of the obtained image data.

The method 100 may substantially culminate, as shown in step 114, in electronically displaying one or more indicia responsive to the conditional state on an electronic display to the user. Such display of indicia may be presented in combination with a portion of the application GUI screen showing adjacent to opaque or semi-opaque indicia displayed according to embodiments.

Referring to step 102, obtaining image data from a computer application corresponding to a first user interface may include obtaining image data including PHI or an analogous field in another application. In step 110, comparing data corresponding to the obtained image data to the corresponding reference database to determine a conditional state corresponding to at least a portion of the obtained image data may include determining a data field including a PHI field or analogous field that is not needed to be known by a party entering other data into the computer application, according to configuration data that may typically be managed by a human operator acting upon knowledge of function(s) typically recorded by a given user or at a given workstation. The inventors contemplate supervisor enablement, for example as to override a preset permissions profile.

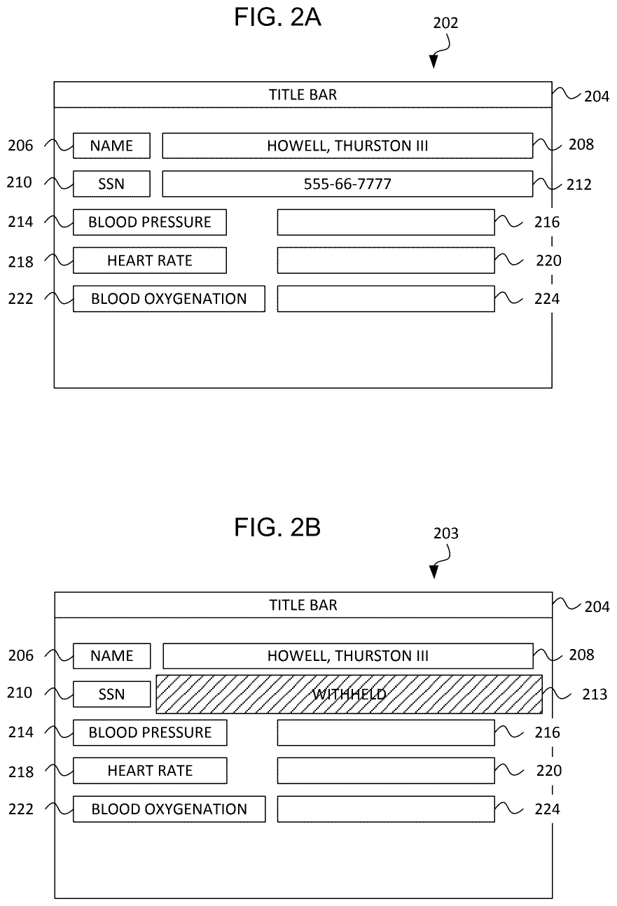

In an embodiment, step 114 may include displaying the one or more indicia responsive to the conditional state on the electronic display to the user, which may include displaying the obscuration indicia instead of a PHI field or other sensitive data field on the electronic display to the user. In an embodiment, displaying the one or more indicia in step 114 includes displaying, on the electronic display, a portion of the first user interface and displaying the one or more indicia over or merged with the first user interface. FIG. 2A diagrammatically illustrates an example of an application display screen 202. FIG. 2B diagrammatically illustrates an example of an augmented application display screen 203, according to an embodiment.

In an embodiment, step 114 may include displaying the one or more indicia on the electronic display, a portion of the first user interface and displaying the one or more indicia registered to an input field or control of the first user interface. In an embodiment, step 114 may include displaying, on the electronic display, a portion of the first user interface and displaying the one or more indicia registered to an input field or control of the first user interface.

Referring again to step 102, obtaining image data, from a computer application, corresponding to a first user interface for display by an electronic display may include performing capture of a momentary display produced by a computer application to create a screen-scraped image.

FIG. 1B is a flow chart showing the computer method 100 of FIG. 1A with additional detail, according to embodiments. Referring to FIG. 1B, a computer method 100B (or 100, 100A, 100C) may include, in step 104, thinning the obtained image data to produce thinned image data. Thinning the obtained image data allows for processing to be performed with reduced computer and/or network resources than with non-thinned image data. In an embodiment where image thinning is performed, comparing data corresponding to the obtained image data in step 110 to the corresponding reference database may include comparing the thinned image data to the corresponding reference database. Thinning the obtained image data may include removing image data that is peripheral to fillable fields in the image data. Thinning the obtained image data in step 104 may include converting fillable fields from a graphical representation to a character-based representation, such as ASCII or Unicode. Additionally or alternatively, thinning the obtained image data in step 104 may include only obtaining image data when the image data changes.

The computer method 100 may further include step 106, in which the thinned image data is encrypted to produce encrypted image data. According to embodiments, the corresponding reference database includes corresponding encrypted reference data. Comparing data corresponding to the obtained image data to the corresponding reference database in step 110 includes comparing the encrypted image data to the corresponding encrypted reference data. In other words, the encryption of the image data and the corresponding reference database may be performed using the same encryption algorithm, thus making identical the encrypted image data and encrypted reference data if the source data was identical. Encryption may be performed using conventional password encryption and/or, additionally or alternatively, using asymmetric public key encryption. According to embodiments herein, it is intended that encryption refers to encryption in addition to conventional JSON web token (JWT) or other token encryption, which may be used for maintaining encrypted communication. Encryption described herein may result in substantially no unencrypted PHI being maintained in an accessible non-transitory storage medium by an augmentation application described herein.

Referring most specifically to FIG. 1B, the method 100B may include, in step 108a, uploading the encrypted image data to a network resource. Accordingly, the corresponding reference database and/or corresponding encrypted reference data may accessed by the network resource. Comparing data corresponding to the obtained image data to the corresponding reference database to determine the conditional state corresponding to at least a portion of the obtained image data, in step 110, is performed by the network resource including comparing the encrypted image data to the corresponding encrypted reference data.

Referring most specifically to FIG. 1C, the method 100C may include, in step 108b, downloading the encrypted reference data from a network resource. Accordingly, the corresponding reference database and/or corresponding encrypted reference data may accessed by the a local computing device. Comparing data corresponding to the obtained image data to a corresponding reference database to determine a conditional state corresponding to the at least a portion of the obtained image data, in step 110, may be performed by the local computing device configured to drive the electronic display.

Referring again to FIGS. 1A, 1B, and 1C, the computer method 100 may include step 112, wherein the one or more indicia corresponding to the conditional state is(are) determined. Comparing data corresponding to the obtained image data to the corresponding reference database to determine the conditional state corresponding to the at least a portion of the obtained image data in step 110 may include determining that the portion of the obtained image data corresponds to protected health information (PHI) that is not required by the user in a current context. The one or more indicia determined in step 112 may include an obscuration pattern; and displaying the one or more indicia responsive to the conditional state, in step 114, may include displaying the obscuration pattern registered to and over the PHI that is not required by the user in the current context.

Referring to FIGS. 2A and 2B, a fillable form 202 displayed by a HIPAA-restricted application, may be identified by information such as a window title bar 204, a form layout of data labels 206, 210, 214, 218, 220 and/or corresponding fillable fields 208, 212, 216, 224, or other information received by the augmentation system. A conditional state for field 212 may correspond to private information (in the figure, a patient social security number) shown in data field 212. Referring to FIG. 2B, the augmentation system may display the obscuration pattern 213 over or in place of the data field 212.

Additionally or alternatively, comparing data corresponding to the obtained image data to the corresponding reference database to determine the conditional state corresponding to the at least a portion of the obtained image data in step 110 may include determining that the portion of the image data corresponds to a data field value that does not match a corresponding reference data field value. Accordingly, the one or more indicia determined in step 112 may include a data mismatch alert pattern; and electronically displaying the one or more indicia responsive to the conditional state, in step 114, may include displaying the data mismatch alert pattern registered to and adjacent to the portion of the obtained image data corresponding to the data field value that does not match the corresponding reference data field value.

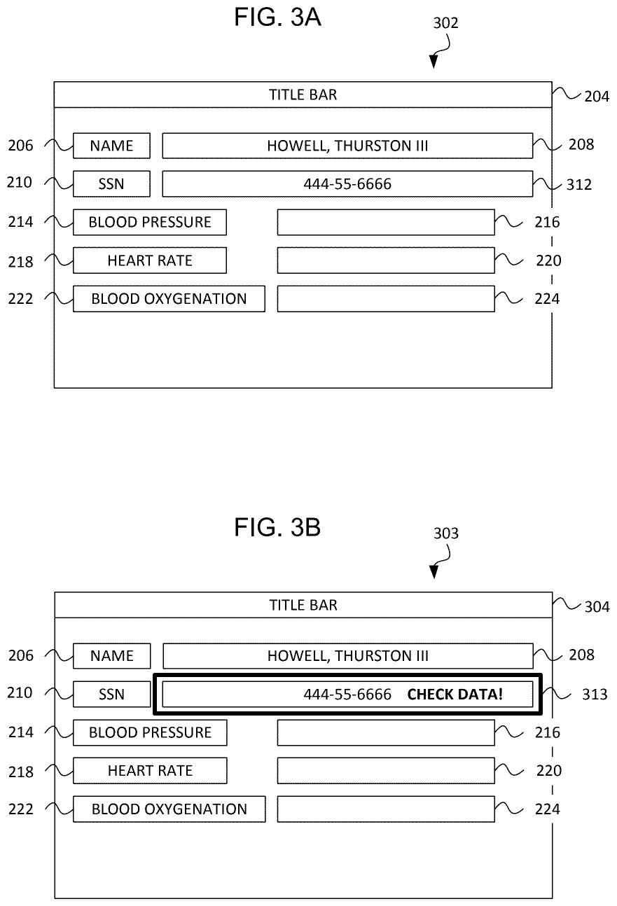

Referring to FIG. 3A in comparison to FIG. 2A, one may see that PHI data (in this example, a patient's social security number 312) entered by a user does not match the correct social security number 212 (FIG. 2A) corresponding to reference data. Referring to FIG. 3B, the augmentation system and corresponding method 100 described herein may cause a mismatch alert pattern 313 displayed adjacent to, in place of, or over the incorrectly entered data 312.

Additionally or alternatively, comparing data corresponding to the obtained image data to the corresponding reference database to determine the conditional state corresponding to the portion of the image layer in step 110 may include determining that the portion of the obtained image data corresponds to a data field that is required to be provided by the user and that a preferred practice suggests filling fields in a preferred order. The one or more indicia determined in step 112 may accordingly include a guided form completion pattern (see, for example, 404, 406). Accordingly, electronically displaying the one or more indicia responsive to the conditional state in step 114 may includes displaying the guided form completion pattern 404, 406 registered to and adjacent to the portion of the image data, respectively corresponding to the data field values 224, 220 that is (are) required to be provided by the user and which is (are) preferred to provided next.

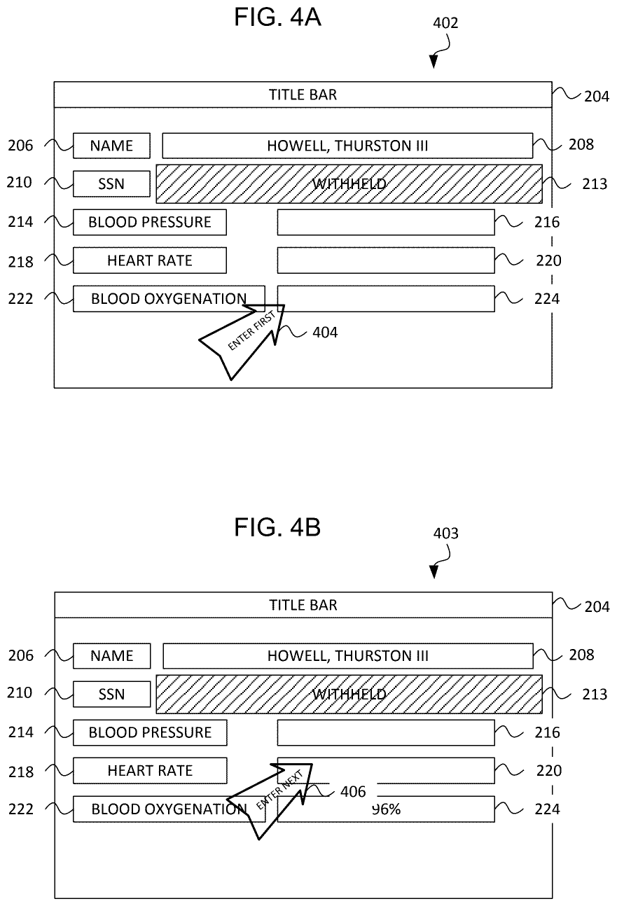

Referring to FIGS. 4A and 4B, a user such as a nurse or technician tasked with taking and recording vital signs may view an application window 402 for recording the vital signs. Supposing, for example, blood pressure and heart rate, labeled as 214 and 218, are taken by automated blood pressure equipment that takes some time to operate, while blood oxygenation is taken by another instrument that obtains its data more quickly. Augmentation may include first displaying a guided form completion pattern 404, indicating that the blood oxygenation should be entered into the fillable field 224 first, owing to the reduced delay to determine blood oxygenation while the automated blood pressure equipment is working on determining that data. After entering the blood oxygenation (indicated to have a value of 96%) in the field 224, the augmentation system and method then displays a guided form completion pattern 406 indicating that heart rate should next be entered into the appropriate field 220. Other, for example, more obscure guidance or more complex, navigation may be readily envisioned. The order of filling out the form may optionally be determined by total time to fill the form 402, 403 according to other previous instances of use, with preferred orders being associated with reduced form filling durations.

Returning to FIGS. 1A, 1B, and 1C, step 110 of comparing data corresponding to the obtained image data to the corresponding reference database may include comparing data corresponding to the obtained image data to a reference database corresponding to a same application display screen as the obtained image data corresponds to. Additionally or alternatively, comparing data corresponding to the obtained image data to the corresponding reference database in step 110 may include comparing data corresponding to the obtained image data to a reference database corresponding to a plurality of application display screens. For example, comparing data corresponding to the obtained image data to the corresponding reference database in step 110 may include comparing data corresponding to the obtained image data to a reference database corresponding to at least one respective display screen from a plurality of computer applications. For example accuracy of an entry made by a user may be determined from reference data corresponding to other data entry windows of the same application and/or corresponding to data entry windows of different applications, when the applications receive or display corresponding fillable fields.

Obtaining the image data in step 102 may be performed in a variety of ways. For example, obtaining the image data in step 102 may include reading the image data from a display buffer. In another embodiment, obtaining the image data in step 102 may include receiving the image data into a display wedge that receives the image data from a local computing device via a display cable and outputs transformed image data to an electronic display. In another example, obtaining the image data in step 102 may include receiving the image data into memory via hardware that mimics a mirrored display screen. Additionally or alternatively, obtaining the image data in step 102 may include capturing a momentarily displayed screen with a digital camera. In another embodiment, obtaining the image data in step 102 includes receiving the image data in a form of a display list, for example, in a specification or display list for a display window. Obtaining the image data in step 102 may include receiving the image data via an Accessibility application programming interface (API), such as an Android API. Obtaining the image data in step 102 may include performing a screen capture. Performing the screen capture may include capturing an only momentarily displayed application screen.

The computer method 100 may further include keylogging a local computing device (step not shown in FIGS. 1A, 1B, and 1C). The keylogged data may be used to infer data entered into an application data field, and from that, infer the application data field identity.

According to an embodiment, the reference data or encrypted reference data includes an artificial intelligence training set, such that comparing data corresponding to the obtained image data to the corresponding reference database in step 110 includes running the artificial intelligence to determine the conditional state.

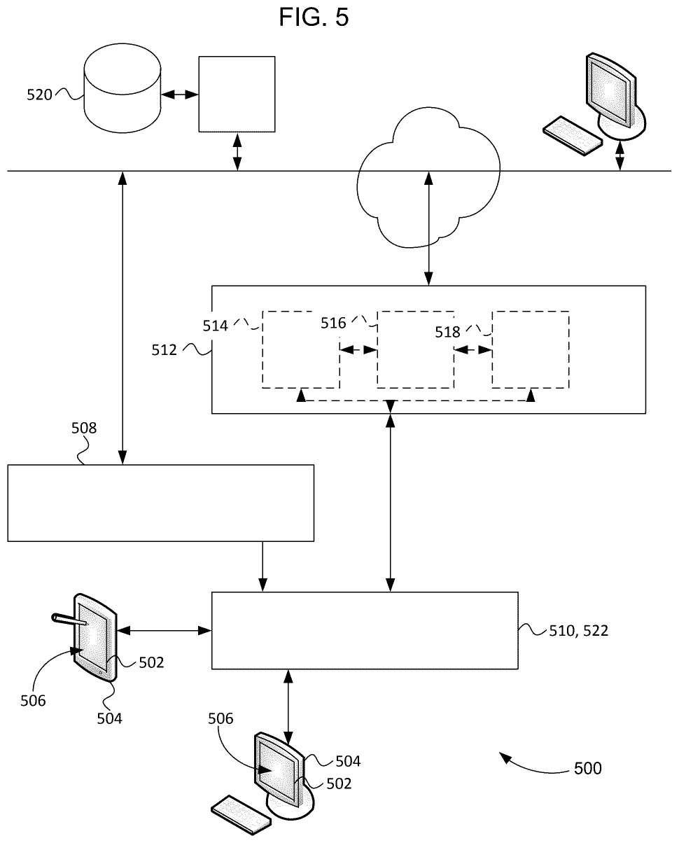

FIG. 5 is a diagram of a computer system 500 configured for providing user-guidance, according to an embodiment. The computer system 500 may include an electronic display 502, driven by a computer 504, configured to display a user interface 506 including at least a portion of a fillable form (See FIGS. 2A, 2B, 3A, 3B, 4A, and 4B) produced by a HIPAA-restricted medical application 508. While the HIPAA-restricted application 508 is illustrated as separate from a microprocessor 512 for executing augmentation and separate from computers 504, it shall be understood that the particular disposition of processing tasks across a single or plural computers is a design choice and various architectures fall within the scope of embodiments described herein. Similarly, as described in more detail elsewhere, the inventors contemplate embodiments where the augmentation application and reference database storage 520 are integrated into computers 502.

A video capture interface 510 may be configured to capture video data including at least a portion of the displayed fillable form. As described elsewhere, the video capture interface is contemplated to take a variety of forms.

A microprocessor 512 may be configured to execute computer-readable instructions corresponding to:
a) an image optimizer 514 configured to receive the captured video data from the video capture interface 510, the image optimizer 514 being configured to determine context and content of the at least the portion of the fillable form and to thin the video data to include at least one graphical data field of the fillable form;
b) a data reader 516 configured to receive the at least one data field from the image optimizer and perform optical character recognition to convert the at least one graphical data field to at least one corresponding character-type (e.g., ASCII or Unicode) data field; and
c) a data analyzer 518 configured to receive the at least one character-type data field from the data reader 516, access a database 520 carrying corresponding reference data field information, compare the at least one character-type data field to the corresponding reference data field information, and output augmentation data responsive to the at least one comparison.

A graphics processor 522, may be configured to receive the augmentation data from the data analyzer 518 and to operate a display engine configured to receive the augmentation data and to drive the electronic display 502 to display an augmentation image adjacent to or over the at least one graphical data field on the electronic display.

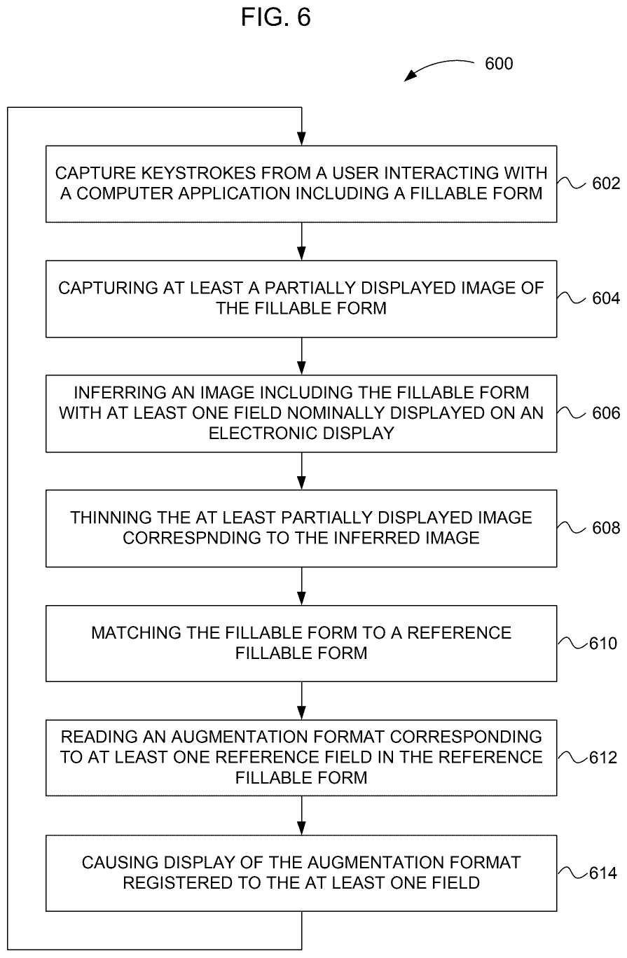

FIG. 6 is a flowchart showing a computer method 600 for causing display of a format relative to at least one field in a fillable form, according to an embodiment. The computer method 600 may include, in step 602, capturing keystrokes from a user interacting with a computer application and, in step 606 inferring an image including a fillable form including at least one field at least nominally displayed on an electronic display. Proceeding to step 610, the computer method 600 includes matching the fillable form to a reference fillable form stored on a non-transitory computer readable medium, in step 612, reading an augmentation format corresponding to at least one reference field in the reference fillable form, and, in step 614, causing display of the augmentation format registered to the at least one field in the fillable form. For example, the augmentation format may be superimposed over, displayed in place of, or displayed adjacent to the at least one field in the fillable form.

The method 600 may include, in step 604, capturing an at least partially displayed image corresponding to the inferred image. The at least partially displayed image may be thinned to reduce an amount of computer memory needed to hold, or bandwidth needed to transmit, information corresponding to the fillable form. Capturing the at least partially displayed image corresponding to the inferred image may include capturing the image including the fillable form including at least one fillable field.

Reading the augmentation format corresponding to the at least one reference field in step 612 may include reading a highlight pattern, for example. Accordingly, causing display of the augmentation format registered to the at least one field may include causing display of the highlight pattern adjacent to the at least one fillable field. In this way, a user may be visually guided to a fillable field for entering data into the fillable form.

Various approaches to capturing the at least partially displayed image are contemplated and may be varied according to operating system or computer environment abilities and limitations. For example, capturing the at least partially displayed image may include reading a display buffer operatively coupled to the electronic display. Additionally or alternatively, capturing the at least partially displayed image may include receiving a mirrored display. In another embodiment, capturing the at least partially displayed image in step 604 includes capturing an image of the electronic display with a focal plane imager.

Referring to step 608, thinning the data may include performing image processing to convert the at least one field to a character string. Matching the fillable form or thinned fillable form to a reference fillable form may include comparing the character string to a corresponding reference character string.

Reading the format corresponding to at least one reference field in the reference fillable form, in step 612 may include performing a Boolean function to determine that the character strings do not match. If the character strings do not match, reading the format in step 612 may include reading a highlighting pattern specified to be displayed over or adjacent to the at least one graphical field, the highlighting pattern being selected to alert the user to the non-match of information displayed in the least one graphical field to information from the reference field.

In an embodiment, reading the format in step 612 corresponding to at least one reference field in the reference fillable form includes performing a Boolean function to determine that the character strings match and reading, for matching character strings, a format including a null pattern, the null pattern being selected to not alert the user.

In an embodiment, the computer method 600 may include encrypting the thinned data (not shown in FIG. 6). In this case, reading the format corresponding to the at least one reference field in the reference fillable form, in step 612, may include comparing the encrypted thinned data to encrypted reference data.

In an embodiment, reading the format corresponding to the at least one reference field in step 612 includes reading an obscuration pattern and causing display of the augmentation format registered to the at least one field in step 614 includes causing display of the obscuration pattern over or in place of the at least one field. In this way, personally identifiable information carried by the at least one field corresponding to a person referenced by the fillable form is obscured from visibility to a user not needing the personally identifiable information to fill out the fillable form. The personally identifiable information may include PHI. Thus, causing display of the augmentation format registered to the at least one field may include obscuring the at least one nominally displayed field.

By encrypting the information displayed in the fillable form or included in the reference field, specific personally identifiable information is not placed in the reference fillable form corresponding to the specific person. The combination of masking sensitive information (such as social security number or HIPAA-constrained information) to view by a user (such as a user not needing access to the sensitive information), encrypting data before transmitting to a network resource for matching and comparison, and maintaining reference data in encrypted form protects a person whose information is a subject of the form.

In one embodiment, the process of extracting user interface data captured from images many be enhanced (faster, more accurate, etc.) by utilizing other non-image information obtained from the operating system, such as keystrokes, the content of the copy buffer, mouse click timing or locations, window titles or header bar information.

In one embodiment, the process of capturing data about the user interface (location of buttons, field and buttons, the lables of those fields, menus or buttons, menu options, field content, User interface choices available to the user and information about which options they have selected, etc.) may be obtained directly from the operating system. In those cases, the user interface may be interpreted and augmented without relying on image information. In Android devices, for example, the Accessibility API functionality of the operating system makes available user interface information in order to assist users with special needs. With the data provided by these types of operating system services, the user interface for special needs users or any user.

One embodiment related to applications is to use an inspection of the underlying code (HTML, JAVA, etc.) to assist with the interpretation of image data, such as code from web applications. Alternatively, inspection of code can be used in conjunction with the interpretation of image data to more efficiently capture data for those applications where it is possible, while the image-based approach is used on other applications where the underlying code is not available.

Some HIPAA-restricted health management computer (HHM) systems and other sensitive systems are designed to operate on Citrix or other virtual machine environments. One embodiment is to use image-based augmentation in the context of applications running on these virtual environments.

Referring again to FIG. 1A, obtaining image data corresponding to a first interface from a computer application, in step 102, may be restated as obtaining user interface data from the computer application corresponding to the first user interface. Step 110 may thus be restated as comparing the obtained user interface data to a corresponding reference database to determine a conditional state corresponding to at least a portion of the obtained user interface data. The method may then proceed through electronically displaying the one or more indicia responsive to the conditional state on the electronic display to the user, in step 114.

Obtaining the user interface data in step 102 may include receiving user interface data via an accessibility application programming interface (API). For example, obtaining the user interface data may include receiving user interface data via automatically reading code in a web application user interface.

As indicated above, the inventors note that a computer application to be augmented may run on a virtual machine environment. In particular, the computer application corresponding to the first user interface may run on a Citrix virtualization. Obtaining the user interface data from the computer application corresponding to the first user interface may include reading a window specification from the Citrix virtualization. CITRIX is available from Cloud Software Group, Inc. of Fort Lauderdale, Florida, USA.

Electronically displaying the one or more indicia responsive to the conditional state on the electronic display to the user may include inserting a substitute window specification into the Citrix virtualization, such that the one or more indicia are included in the substitute window specification. Inserting the substitute window specification into the Citrix virtualization may include removing data from the window specification from the Citrix virtualization. Inserting the substitute window specification into the Citrix virtualization may, additionally or alternatively include adding data to the window specification from the Citrix virtualization. In an embodiment, inserting the substitute window specification into the Citrix virtualization includes replacing data corresponding to a field from the window specification from the Citrix virtualization.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

What is claimed is:

1. A computer method for augmenting a graphical user interface of a computer application, comprising:
obtaining image data from a computer application corresponding to a first user interface;
comparing data corresponding to the obtained image data to a corresponding reference database to determine a conditional state corresponding to at least a portion of the obtained image data; and
electronically displaying one or more indicia responsive to the conditional state on an electronic display to the user,
wherein comparing the data corresponding to the obtained image data to the corresponding reference database to determine a conditional state corresponding to at least a portion of the obtained image data includes determining that the at least a portion of the obtained image data corresponds to protected information that is not required by the user in a current context.

2. (canceled)

3. The computer method for augmenting the graphical user interface of claim 1:
wherein displaying the one or more indicia includes displaying, on the electronic display, a portion of the first user interface and displaying the one or more indicia over or merged with the first user face.

4. (canceled)

5. The computer method for augmenting the graphical user interface of claim 1:
wherein displaying the one or more indicia includes displaying, on the electronic display, a portion of the first user interface and displaying the one or more indicia registered to an input field of the first user interface.

6. The computer method for augmenting the graphical user interface of claim 1:
wherein obtaining image data includes performing capture of a momentary display produced by the computer application to create a screen-scraped image.

7. The computer method for augmenting the graphical user interface of claim 1, further comprising:
thinning the obtained image data to produce thinned image data;
wherein comparing the data corresponding to the obtained image data to the corresponding reference database includes comparing the thinned image data to the corresponding reference database.

8. The computer method for augmenting the graphical user interface of claim 7, wherein thinning the obtained image data includes at least one of removing image data that is peripheral to fillable fields in the image data, converting fillable fields from a graphical representation to a character-based representation, and only obtaining image data when the image data changes.

9.-10. (canceled)

11. The computer method for augmenting the graphical user interface of claim 7, further comprising:
encrypting the thinned image data to produce encrypted image data;
wherein the corresponding reference database includes corresponding encrypted reference data; and
wherein comparing the data corresponding to the obtained image data to the corresponding reference database includes comparing the encrypted image data to the corresponding encrypted reference data.

12. The computer method for augmenting the graphical user interface of claim 11, further comprising:
uploading the encrypted image data to a network resource;
wherein the corresponding reference database and corresponding encrypted reference data are accessed by the network resource; and
wherein comparing the data corresponding to the obtained image data to the corresponding reference database to determine a conditional state corresponding to at least a portion of the obtained image data is performed by the network resource including comparing the encrypted image data to the corresponding encrypted reference data.

13. The computer method for augmenting the graphical user interface of claim 11, further comprising:
downloading the encrypted reference data;
wherein comparing the data corresponding to the obtained image data to a corresponding reference database to determine a conditional state corresponding to the at least a portion of the obtained image data is performed by a local computing device configured to drive the electronic display.

14. (canceled)

15. The computer method for augmenting the graphical user interface of claim 1:
wherein the protected information is protected health information (PHI);
wherein the one or more indicia includes an obscuration pattern; and
wherein displaying the one or more indicia responsive to the conditional state includes displaying the obscuration pattern registered to and over or in place of the PHI that is not required by the user in the current context.

16. The computer method for augmenting the graphical user interface of claim 1:
wherein comparing the data corresponding to the obtained image data to the corresponding reference database to determine the conditional state corresponding to the at least a portion of the obtained image data includes determining that the portion of the image data corresponds to a data field value that does not match a corresponding reference data field value;
wherein the one or more indicia determined includes a data mismatch alert pattern; and
wherein electronically displaying the one or more indicia responsive to the conditional state includes displaying the data mismatch alert pattern registered to and adjacent to the portion of the obtained image data corresponding to the data field value that does not match the corresponding reference data field value.

17. The computer method for augmenting the graphical user interface of claim 1:
wherein comparing the data corresponding to the obtained image data to the corresponding reference database to determine the conditional state corresponding to the portion of the image data includes determining that the portion of the obtained image data corresponds to a data field that is required to be provided by the user and that a preferred practice corresponds to filling fields in a preferred order;
wherein the one or more indicia one or more indicia includes a guided form completion pattern; and
wherein electronically displaying the one or more indicia responsive to the conditional state includes displaying the guided form completion pattern registered to and adjacent to the portion of the image data corresponding to the data field value that is required to be provided by the user and that the preferred practice indicates should be filled in a given order.

18. (canceled)

19. The computer method for augmenting the graphical user interface of claim 1:
wherein comparing the data corresponding to the obtained image data to the corresponding reference database includes comparing the data corresponding to the obtained image data to a reference database corresponding to a plurality of application display screens.

20. The computer method for augmenting the graphical user interface of claim 1:
wherein comparing the data corresponding to the obtained image data to the corresponding reference database includes comparing the data corresponding to the obtained image data to a reference database corresponding to at least one respective display screen from a plurality of computer applications.

21. The computer method for augmenting the graphical user interface of claim 1, wherein obtaining the image data includes reading the image data from a display buffer.

22.-25. (canceled)

26. The computer method for augmenting the graphical user interface of claim 1:
wherein obtaining the image data includes receiving user interface data via an accessibility application programming interface (API);
wherein the user interface data includes at least one selected from the group consisting of one or more field names, one or more field locations, one or more button locations and labels, one or more menu options, and one or more instances of field content.

27. The computer method for augmenting the graphical user interface of claim 1, wherein obtaining the image data includes performing a screen capture.

28. (canceled)

29. The computer method for augmenting the graphical user interface of claim 1, further comprising:
keylogging a local computing device; and
using the keylogged data to infer data entered into an application data field.

30. The computer method for augmenting the graphical user interface of claim 1:
wherein the reference data or encrypted reference data includes an artificial intelligence training set; and
wherein comparing data corresponding to the obtained image data to the corresponding reference database includes running the artificial intelligence to determine the conditional state.

31. A computer system for providing user-guidance, comprising:
an electronic display, driven by a compute, configured to display a user interface including at least a portion of a fillable form produced by a HIPAA-restricted medical application;
a video capture interface configured to capture video data including at least a portion of the displayed fillable form;
a microprocessor configured to execute computer-readable instructions corresponding to:
an image optimizer configured to receive the captured video data from the video capture interface, the image optimizer being configured to determine context and content of the at least the portion of the fillable form and thin the video data to include at least one graphical data field of the fillable form;
a data reader configured to receive the at least one graphical data field from the image optimizer and perform optical character recognition to convert the at least one graphical data field to at least one corresponding character-type data field; and
a data analyzer configured to receive the at least one character-type data field from the data reader, access a database carrying corresponding data field information, compare the at least one character-type data field to the corresponding data field information, and output augmentation data responsive to the at least one comparison; and
a graphics processor configured to receive the augmentation data from the data analyzer and to operate an embedded display engine configured to receive the augmentation data and to drive the electronic display to display an augmentation image adjacent to or over the at least one graphical data field on the electronic display.

32. A computer method, comprising:
capturing keystrokes from a user interacting with a computer application;
inferring, from content of the keystrokes, an image including a fillable form including at least one field at least nominally displayed on an electronic display;
matching the fillable form to a reference fillable form stored on a non-transitory computer readable medium;
reading a format corresponding to at least one reference field in the reference fillable form; and
causing display of the format registered to the at least one field.

33. The computer method of claim 32, further comprising:
capturing an at least partially displayed image corresponding to the inferred image; and
thinning the at least partially displayed image to reduce an amount of computer memory needed to hold information corresponding to the fillable form.

34.-41. (canceled)

42. The computer method of claim 32:
wherein reading the format corresponding to the at least one reference field includes reading an obscuration pattern; and
wherein causing display of the format registered to the at least one field includes causing display of the obscuration pattern over or in place of the at least one field;
whereby personally identifiable information carried by the at least one field corresponding to a person referenced by the fillable form is obscured from visibility to a user not needing the personally identifiable information to fill out the fillable form.

43. The computer method of claim 42, wherein the personally identifiable information includes protected health information (PHI).

44.-51. (canceled)
